(19) United States
(12) Patent Application Publication
(10) Pub. No.: US 2003/0032649 A1
(43) Pub. Date: Feb. 13, 2003

(54) Chimerizing protein kinases for drug discovery

(76) Inventors: Elizabeth J. Goldsmith, Dallas, TX (US); Akella Radha, Plano, TX (US); Richard B. Gaynor, Dallas, TX (US)

Correspondence Address: BAKER & BOTTS, 30 ROCKEFELLER PLAZA, NEW YORK, NY, 10112

(21) Appl. No.: 09/918,873

(22) Filed: Jul. 31, 2001

(51) Int. Cl. C12N 9/12; A61K 31/52; G06F 19/00; G01N 33/48; G01N 33/50

(52) U.S. Cl. 514/263.1; 435/194; 702/19; 514/46

(57) ABSTRACT

The present invention relates to chimeric protein kinase molecules and methods for designing inhibitors of protein kinases using the chimeric protein kinases of the present invention. The chimeric protein kinase of the present invention comprise inhibitor binding site residues of a non-crystallizable protein and non-inhibitor binding site residues of a crystallizable protein. The chimeric protein is preferably crystallizable and is useful for designing inhibitors for the non-crystallizable protein. In addition, the present invention is directed to a protein kinase inhibitor binding site which is outside the ATP binding site of the protein kinase and methods of use therefore.

This invention was made in part with government support under NIH Grant Number DK46993. Therefore, the government has certain rights in the invention.

### BACKGROUND OF THE INVENTION

The present invention generally relates to methods for designing inhibitors of proteins, and particularly, kinases which are not readily crystallizable and therefore do not lend themselves readily to rational drug design techniques. The present invention is also directed to chimeric protein molecules, and in particular, chimeric kinases which comprise inhibitor binding site residues of a non-crystallizable protein and non-inhibitor binding site residues of a crystallizable protein. The chimeric protein is crystallizable and is useful for designing inhibitors for the non-crystallizable protein, which interact with its inhibitor binding site. In addition, the present invention is directed to a protein kinase inhibitor binding site which is outside the ATP binding site of the protein kinase and methods of use therefore. The protein kinase inhibitor binding site which is outside the ATP binding site of the present invention is useful, inter alia, for designing protein kinase inhibitors.

Protein kinases are a family of diverse, but related enzymes which exhibit a unique catalytic function. They play a role in virtually all regulatory processes ranging from ion transport to metabolic pathways to DNA replication and differentiation. See Hanks and Hunter, Science, 241:42-52 (1988) and Krebs, Biochems. Soc. Trans, 13:813-820 (1985). Mitogen activated protein (MAP) kinases, for example, are involved in signal transduction pathways associated with cellular processes such as cell proliferation, response to environmental stress and cell death. See Lewis, Shapiro and Ahn, Adv. Cancer Res. 74:49-139 (1998). MAP kinases and their upstream activators, MEKS, have been implicated in signaling pathways in several disease pathways. Nonlimiting examples of diseases and disease pathways in which protein kinases have been implicated include apoptosis [Anderson, Microbiol. Mol. Biol. Rev., 61:33-46 (1997)], cancer [Dirks, Neurosurgery, 40:1000-13, (1997); Brunton and Workman, Cancer Chemother. Pharmacol. 32:1-19 (1993); Powis, Pharmacol. Ther. 62:57-95 (1994)], Alzheimer's disease [Imahori et al., J. Biochem., 121:179-88 (1997)] angiotensin II and hematopoietic cytokine receptor signal transduction [Berk et al., Circ. Res., 80:607-16 (1997); Mufson, FASEB J., 11:37-44 (1997)], oncoprotein signaling and mitosis [Laird et al., Cell Signal, 9:249-55 (1997)], inflammation and infection [Han et al., Nature, 386:296-9 (1997)], rheumatoid arthritis [Badger et al., J Pharmacol. Exp. Ther. 279:1453-1461 (1996)], and psoriasis [Elder et al., Science 243:811-814 (1989)].

Due to the regulatory role of protein kinases, many protein kinases have been targeted for drug discovery. Technological advances in areas such as structural characterization of biomacromolecules, computer sciences and molecular biology have made rational drug design more feasible. See Ooms, Curr. Med. Chem 7:141-158 (2000); Gane and Dean, Curr. Opin. Struct. Biol. 10:401-404 (2000). Therefore, a structural understanding of the inhibition of kinase activity could lead to the discovery of new inhibitory molecules useful in the treatment of disease.

The structures of a number of protein kinases have been solved by X-ray diffraction and analyzed [reviewed by Johnson et al., Cell, 85:149-158 (1996); Goldsmith et al., Cur. Opin. Struct. Biol., 4:833-840 (1994); Taylor et al., Structure, 2:345-355 (1994)]. The structure of enzymes of the kinase family is characterized by two domains separated by a deep cleft. The N-terminal domain and cleft create a binding pocket for the adenine ring of ATP, and the C-terminal domain contains the presumed catalytic base, magnesium binding sites, and phosphorylation site. While sequence homology among the kinases varies, the three-dimensional structure of kinases remains very related and both the sequence homology and the structural homology are usually highest in the ATP-binding site.

Among medically important tyrosine kinases are epidermal growth factor receptor (EGFR), platelet-derived growth factor receptor (PDGFR), fibroblast growth factor receptor (FGFR), Flk-1, and src. Medically important serine/threonine kinases include IKKβkinase, NIK (a Ste 20 homolog), Akt (protein kinase B), glycogen synthase kinase-3 (GSK-3), MAPKAP, and others. One particularly important class of serine/threonine kinases are the mammalian mitogen-activated protein (MAP) 1 kinases. These kinases mediate intracellular signal transduction pathways [Cobb et al., J. Biol. Chem., 270:14843-6 (1995)]. Members of the MAP kinase family share sequence similarity and conserved structural domains, and include the extracellular-signal regulated kinases (ERKs), Jun N-terminal kinases (JNKs) and p38 kinases.

JNK and p38 kinases are activated in response to the pro-inflammatory cytokines TNF-α and interleukin-1, and by cellular stress such as heat shock, hyperosmolarity, ultraviolet radiation, lipopolysaccharides and inhibitors of protein synthesis [Derijard et al., Cell, 76: 1025-37 (1994); Han et al., Science, 265:808-11 (1994); Raingeaud et al., J. Biol. Chem., 270:7420-6 (1995); Shapiro et al., Proc. Natl. Acad. Sci. U.S.A., 92:12230-4 (1995)]. Also involved in the inflammatory response is IκB kinase (IKK-B) which phosphorylates IκB, the inhibitory protein of the transcription factor NF-κB. NF-κB is involved in the regulation of the inflammatory response [Baeuerle and Henkel. Ann Rev Immunol. 12, 141-79 (1994); Ghosh et al. Ann Rev Immunol. 16, 225-60 (1998)].

Another family of protein kinases involved in cell cycle regulation and implicated in a number of diseases are the cyclin-dependent kinases. The eukaryotic cell cycle is coordinated by several related serene/threonine protein kinases, each consisting of a catalytic cyclin-dependent kinase (CDK) subunit and a regulatory cyclin subunit. This family of protein kinases drives cell cycle events such as cell growth, DNA replication and cell division [see Sherr, Cell 79:551-555 (1994); Heichman & Roberts, Cell 79:557-562 (1994); Sobczak-Thepot Expl. Cell. Res. 206:43-48 (1993) and King, Jackson and Kirschner, Cell 79:563-571 (1994)]. Structural characterization of cyclin-dependent kinases in inactive, active and inhibited forms has revealed the mechanism by which this family of protein kinases are regulated. See Jeffrey et al., Nature 376:313-320 (1996); Russo et al., Nature 395:237-243 (1997); Russo et al., Nature 382:325-331; and Russo, Jeffrey & Pavletich, Nature Struct. Biol. 3:696-700 (1996). These structures have indicated that regulation of kinase activity involves movement of the two lobes of the kinase in relation to each other along a “hinge” which serves to block (when in a “closed” confirmation) or make accessible (when in an “open” conformation) the ATP binding pocket. In addition, naturally occurring protein inhibitors cause changes in the kinase when bound to the kinase which either (1) effect the structure of the kinase, (2) block the ATP binding pocket and/or (3) block the substrate binding site.

The crystal structures of ERK2 [Zhang et al., Nature, 367, pp. 704-11 (1994); (Brookhaven PDB entry, 1ERK)], unphosphorylated p38 [Wilson et al., J. Biol. Chem., 271:27696-700 (1996); Wang et al., Proc. Natl. Acad. Sci. U.S.A., 94:2327-32 (1997);(Brookhaven PDB entry, 1 WFC)], and a phosphorylated ERK2 have also been solved [Canagarajah et al., Cell, 90:859-69 (1997)].

Known crystal structures of protein kinases reveals that they share very similar fold and topology and are structurally homologous. This is true even though the amino acid sequences of protein kinases may be very divergent, i.e., low sequence homologly or identity.

p38 was identified as a kinase that was phosphorylated on tyrosine and threonine following stimulation of monocytes by LPS [J. C. Lee et al., Nature, 372, pp. 739-46 (1994)]. p38 kinase has been cloned [Han et al., Science, 265:808-11. (1994)] and shown to be the target for pyridinylimidazole compounds that block the production of IL-1β and TNF-α by monocytes stimulated with LPS [Lee et al., Nature, 372:739-46 (1994)]. SB203580, a 2,4,5-triarylimidazole, is a potent p38 kinase inhibitor that is selective relative to other kinases, including other closely related MAP kinases [Cuenda et al., FEBS Lett. 364:229-33 (1995); Cuenda et al., EMBO J., 16:295-305 (1997)]. The structure of SB203580 in complex with p38 has been reported [Tong et al., Nat. Struct. Biol., 4:311-6(1997) and Wang et al. Structure 6, 1117-28 (1998)]. The crystal structure of a different pyridinylimidazole compound, VK-19,911, 4-(4-fluorophenyl)-1-(4-piperidinyl)-5-(4-pyridyl)-imidazole in complex with p38 has also been described [Wilson et al., Chem. & Biol. 4:223-231 (1997)]. These structures identified the residues important for binding pyridinyl-imidazoles, and revealed that both compounds bind within the ATP binding site of p38. Many of these residues are conserved in ERK2 as well, but there are enough differences that binding of pyridinyl-imidazole compounds does not occur. A similar situation exists for JNK3, which also shares structural similarity to p38, but is unable to bind pyridinyl-imidazole inhibitors. This same type of scenario, wherein a compound binds to one family member, but not to the majority of others may occur in other serine/threonine kinase and tyrosine kinase families.

Therefore, it is possible that kinase-specific inhibitors which bind to the ATP binding site can be designed using the understanding of specificity obtained from studying the structure of protein kinases bound to specific inhibitors. However, since there are an estimated 2000 different protein kinases present in human, all of which use ATP as a second substrate, there exists a possibility that inhibitors which bind to the ATP binding site of a particular protein kinase may also, inadvertently and undesirably, bind to and inhibit more than one protein kinase. Therefore, it is desirable to identify inhibitors of protein kinases which do not involve binding to the ATP binding site and which have demonstrate specificity.

While many protein kinase structures have been solved, there are many protein kinases which do not lend themselves to structural determination. There are many reasons why structural determination may not be achievable. For example, one necessary step in the structural determination of a protein is to express the protein in sufficient quantity and in a properly folded state. Some proteins are not easily expressed by methods known in the art. Since structural determination requires a significant amount of protein (usually in excess of 10 mg), low expression levels are not conducive to structural determination. In addition, while some proteins may be expressed at high levels, they may be misfolded or denatured and may not be easily renatured or folded correctly. This also is not conducive to structural determination of the native state of the protein. Furthermore, even after sufficient amounts of a protein are obtained, crystallization of proteins is a difficult art and represents a third obstacle towards structural determination by x-ray crystallography. Lastly, even if crystals are obtained, they may not be well ordered and may not diffract to a resolution that allows structural determination or they may not otherwise be useful for structural determination (e.g. they are not easily derivitizable and therefore, the structure cannot be solved).

U.S. Pat. No. 6,162,613 of Su et al., (the “'613 patent”) relates to a method for designing inhibitors of serine/threonine protein kinases and tyrosine protein kinases through the use of ATP binding site mutants of kinases. The '613 patent is directed to a method for designing an inhibitor of a second protein kinase comprising providing a first protein kinase having a known three dimensional structure, identifying amino acids in the ATP binding site of the first protein kinase that forms close contacts with a compound known to bind in the ATP binding site, identifying a second protein kinase through a protein alignment means that includes one or more amino acids that align with, but are different from, the amino acids that form close contacts with the compound in the first protein kinase, altering the amino acids in the second kinase that align with the amino acids that form the close contacts to provide a mutant second kinase which includes ATP binding site residues from the first (crystallizable) protein kinase and non-ATP binding site residues from the second (non-crystallizable) protein kinase, identifying compounds that bind to the mutant protein kinase with at least 10 fold greater affinity than that of the second protein kinase, using molecular modeling means to determining how to modify the compound to design an inhibitor that binds to the second protein kinase. While this method may be useful for the determination of inhibitor molecules for the second protein kinase, it does not provide insight into the structure of the ATP binding site of the second (non-crystallizable) protein kinase. The structure of the ATP binding site is useful for the rational design of inhibitor molecules of the non-crystallizable protein kinase.

Some inhibitors of protein kinases are non-competitive with ATP. The MAP/ERK kinase inhibitor PD98059 is known to bind at a site outside the ATP site [Favata et al. J. Biol Chem. 273: 18623-32 (1998)]. Furthermore, hydroxynaphthalene derivatives that inhibit the tyrosine kinase pp60^{c-src} have been shown to be non-competitive with ATP [Marsilje et al. Bioorganic and Medicinal Chem. Lett. 10 477-481 (2000)]. The binding site can be identified by crystallography in a crystallizable homolog of the drug target or by other means, such as mutagenic analysis.

The present invention addresses the inadequacies of the prior art by providing a method for identifying protein kinase inhibitors which bind the protein kinase of interest by providing a chimeric molecule comprising non-inhibitor binding site amino acids from a crystallizable protein kinase and inhibitor binding site amino acids from a non-crystallizable protein kinase. This chimeric molecule allows for the structural determination of the inhibitor binding site of an otherwise non-crystallizable protein kinase and is useful in the identification and design of inhibitor binding site-binding inhibitor molecules for a non-crystallizable protein kinase. The present invention is also directed to a protein kinase inhibitor binding site which is outside the ATP binding site and which is useful for the identification and design of protein kinase inhibitors molecules which bind outside the ATP binding site. The inhibitors disclosed in present invention cause conformational changes in the hinge region, activation loop and phosphate binding ribbon. These changes may influence the binding of ATP or substrates to the kinase.

### SUMMARY OF THE INVENTION

The present invention is directed to crystallizable chimeric protein kinases having a binding site which comprises amino acid residues from a crystallizable protein kinase that do not bind to an inhibitor and amino acid residues from a non-crystallizable protein kinase which bind to the inhibitor. The resultant chimeric protein kinase can be crystallized and the structure of the chimeric protein kinase can be solved by x-ray crystallography. The structure of the chimeric protein kinase is useful for the rational drug design of inhibitors of the non-crystallizable protein kinase. In a preferred embodiment, the chimeric protein kinase of the present invention comprises non-inhibitor binding site amino acid residues from a kinase selected from the group consisting of p38, ERK2, Src, CAPK, CK1, EGF-R, CDK2, and inhibitor binding site amino acid residues from IKK-β, Map/ERK, JNK, and MEK, Akt, GSK-3 and NIK. In another preferred embodiment, the chimeric protein kinase of the present invention comprises non-inhibitor binding site residues from p38 and inhibitor binding site residues from IKK-β, Map/ERK, JNK, and MEK. The present invention is also directed to a method for designing an inhibitor of a first protein kinase which comprises making a chimeric protein kinase having an inhibitor binding site amino acid residues from the first protein kinase that bind the inhibitor and amino acid residues from a second, crystallizable protein kinase that do no bind the inhibitor; crystallizing the chimeric protein kinase; solving the three-dimensional structure of the chimeric protein kinase; analyzing the inhibitor binding site of chimeric protein kinase from the three-dimensional protein kinase; designing an inhibitor which binds to the inhibitor binding site of the chimeric protein kinase and determining whether the inhibitor inhibits the first protein kinase.

In addition, the present invention is directed to a protein kinase inhibitor binding site which is outside the ATP binding site. This protein kinase inhibitor binding site which is outside the ATP binding site is useful, inter alia, for the rational design of non-ATP binding site inhibitors of protein kinases. In a preferred embodiment, the inhibitor binding site of the present invention which is outside the ATP binding site comprises an amino acid sequence corresponding to an amino acid sequence of p38 and structurally homologous to a domain of p38 wherein said domain is defined by a start point at linker L5 (residues 76-83) that joins helix C (residues 63-75) with β4 (residues 84-89), the crossover connection (L7) (residues 106-109) an end point at the C-terminus (βL16) (residues 310-336) of p38. This domain has corresponding amino acids in IKK-β, Map/ERK kinase, εJNK, MEK, GSK-3, Akt, and NIK. See FIG. 7. Therefore, a further embodiment of the present invention is a method of designing a protein kinase inhibitor comprising (1) employing the structure of a protein kinase bound to an inhibitor at an inhibitor binding site (wherein the inhibitor binding site may be identified previously or found by screening) (2) applying standard methods of structure-based drug design based using structure of the protein kinase bound to the inhibitor to identify and characterize additional inhibitors directed to the inhibitor binding site.

### BRIEF DESCRIPTION OF THE DRAWINGS

The present invention may be better understood with reference to the attached drawings in which—

FIG. 1 provides a sequence alignment of p38 with (a) IKK-β and (b) MEK1. In bold are the residues which are identical. The low consensus residues are underlined and the neutral residues are in normal type. p38 shares 25-30% identity with IKK-β and MEK1.

FIG. 2 provides a chemical formula of (a) sulindac sulfide and (b) PD98059. The different moieties in the molecules as used in the text are labelled for claity

FIG. 3 provides a close-up depiction of the structure of p38 bound to (a) sulindac sulfide, and (b) PD98059. Also, the dotted lines indicate the hydrogen bonds between the inhibitor molecule and p38.

FIG. 4 provides a depiction of the structure of the inhibitor binding site of the present invention showing the binding of sulindac sulfide and PD98059 is depicted on a p38 molecule and superimposed is the well-known ATP-competitive inhibitor binding site showing the SB203580 molecule. The activation lip is shown in red.

FIG. 5 provides a ribbon diagram of p38 indicating the numbering scheme of the helices and β-strands. The amino acid numbers corresponding to the varios helices and strands in p38 are as follows:

α_{c}(63-77), α_{d}(113-119), αₑ(123-143), α_{f}(203-218), α_{g}(228-238), αₕ(279-289), α_{I}(299-304), β1(25-33), β2(36-43), β3(48-56), β4(87-91), β(101-107), β6(146-150), β7(156-159), β8(163-167), β9(173-177) and C-terminus 345-356. The sequence numbers absent in the list are those of the linkers.

FIG. 6 provides a close-up of superposition of native p38 (blue) and Sulindac bound p38 (green) showing conformational changes in inhibitor binding site and activation loop.

FIG. 7 provides a sequence alignment near the two inhibitor binding sites of p38 with IKKβ, MEK1, JNK-3, GSK-3, Akt and NIK.

### DETAILED DESCRIPTION OF THE INVENTION

In one embodiment, the present invention is directed to a chimeric protein kinase having an inhibitor binding site comprising amino acid residues from a first protein kinase that bind an inhibitor and amino acid residues from a second protein kinase that do not bind the inhibitor. The inhibitor binding site may be the ATP site, the inhibitor binding site of the present invention which is outside the ATP binding site, or another inhibitor binding site. The resultant chimeric protein kinase is crystallizable and useful for designing inhibitors of the first protein kinase. In a preferred embodiment, the first and second protein kinases are selected from the group consisting of serine/threonine protein kinases and tyrosine protein kinases. In another preferred embodiment, the first protein kinase is not easily crystallizable and the second protein kinase is readily crystallizable. In a further preferred embodiment, the first protein kinase is selected from the group consisting of IKK-β, Map/ERK kinase, JNK, MEK, GSK-3, Akt, and NIK and the second protein kinase is selected from the group consisting of p38, ERK2, Src, CAPK, CK1, EGF-R, CDK2, and FGF-R. In another embodiment, the first protein kinase is selected from the group consisting of IKK-β, Map/ERK kinase, JNK, and MEK, and the second protein kinase is p38. In a particularly preferred embodiment, the chimeric protein comprises an ATP binding site having ATP binding site amino acid residues from IKK-β, Map/ERK kinase, JNK, or MEK, and non-ATP binding site amino acid residues from p38. In a second particularly preferred embodiment, the chimeric protein comprises amino acids from an inhibitor binding site from IKK-β, Map/ERK kinase, JNK, or MEK, and non-inhibitor binding site residues from p38. For example, the chimeric protein kinase of the present invention can consist largely of a p38 amino acid sequence having IKK-β ATP binding site amino acids and p38 non-ATP binding site amino acids.

The following is a nonlimiting list of preferred chimeric protein kinases of the present invention: IKKβ/p38 ATP binding site chimera comprising an amino acid sequence of p38 with mutations Tyr 35 to Phe, Leu75 to Met and Thr106 to Glu; MEK1/p38 ATP binding site chimera comprising an amino acid sequence of p38 with mutations Tyr 35 to Gly and Thr106 to Glu; JNK-3/p38 ATP binding site chimera comprising an amino acid sequence of p38 with mutations Tyr 35 to Gln, Leu75 to Met and Thr106 to Met. GSK-3/p38 ATP binding site chimera comprises of p38 with mutations Tyr 35 to Phe, Leu75 to Met and Thr106 to Leu. Akt/p38 ATP binding site chimera comprises p38 with mutations Tyr 35 to Phe and Thr106 to Glu; NIK/p38 ATP binding site chimera comprises p38 with mutations Tyr 35 to Ala, Leu75 to Ser and Thr106 to Val; IKKβ/p38 inhibitor binding site chimera comprises p38 with mutations Lys79 to Asn, Glu81 to Pro His 107 to Tyr and C-terminus (351-356)PPLDQE to KPATQC; MEK1/p38 inhibitor binding site chimera comprising an amino acid sequence of p38 with mutations Lys79 to Asn, Glu81 to Pro and C-terminus (351-356)PPLDQE to THAASI; JNK-3/p38 inhibitor binding site chimera comprising an amino acid sequence of p38 with mutations Lys79 to Asn, Glu81 to Lys and His 107 to Glu; GSK-3/p38 inhibitor binding site chimera comprising an amino acid sequence of p38 with mutations Lys79 to Asp, Glu81 to Cys, His 107 to Asp and C-terminus (351-356)PPLDQE to PHARIQ; Akt/p38 inhibitor binding site chimera comprises p38 with mutations Lys79 to Arg, Glu81 to Pro His 107 to Tyr and C-terminus (351-356)PPLDQE to FPQFSV; NIK/p38 inhibitor binding site chimera comprising an amino acid sequence of p38 with mutations Lys79 to Arg, Glu81 to Val, His 107 to Asn and C-terminus (351-356)PPLDQE to TLAVKE.

In another embodiment, the present invention is directed to a method of designing an inhibitor molecule for a first protein kinase. The first protein kinase may be non-crystallizable. The method for designing an inhibitor molecule for a first protein kinase comprises making a chimeric protein kinase having inhibitor binding site comprising amino acid residues from a first protein kinase that bind an inhibitor and amino acid residues from a second protein kinase that do not bind the inhibitor wherein the chimeric protein kinase is crystallizable, crystallizing the chimeric protein kinase, solving the three-dimensional structure of the chimeric protein kinase, analyzing the inhibitor binding site of the chimeric protein kinase using the three-dimensional structure, designing an inhibitor which binds to the inhibitor binding site of the chimeric protein kinase via molecular modeling to modify a known inhibitor binding site wherein the modified inhibitor binds to the chimeric protein kinase, and determining whether the inhibitor inhibits the first protein kinase. In a preferred embodiment, the first protein kinase is IKK-β and the inhibitor molecule inhibits IKK-β by binding to its binding site, which is described herein as the inhibitor binding site of the present invention.

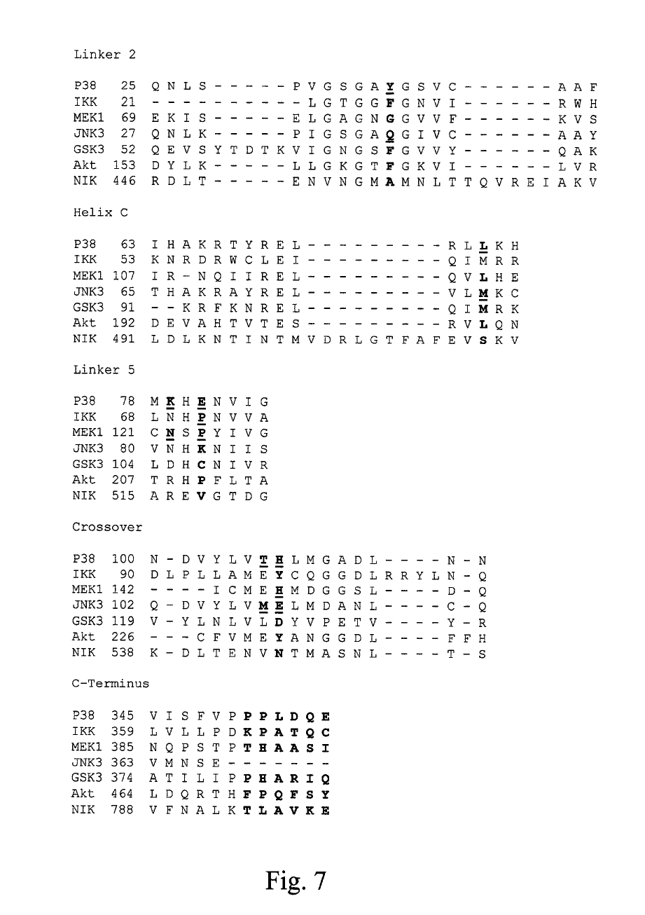

In a further embodiment, the present invention is directed to a protein kinase inhibitor site which corresponds to an amino acid sequence of, and has three-dimensional structural homology to, a domain of p38 starting with the linker L5 (residues 76-83) that joins helix C (residues 63-75) with β4 (residues 84-89), the crossover connection (L7) (residues 106-109) and ending at the C-terminus (βL16) (residues 310-336). This domain has corresponding amino acids in IKK-β, Map/ERK kinase, εJNK, MEK, GSK-3, Akt, and NIK. See FIG. 7. This inhibitor binding site is useful, inter alia, for designing protein kinase inhibitor molecules which do not bind to the ATP binding site of protein kinases. Therefore, the present invention also provides a method for identifying non-ATP binding site inhibitors of protein kinases comprising employing the structure of a protein kinase having an inhibitor bound to amino acids corresponding to amino acids Lys79, Glu 81, His 107, Lys 165, and the C-terminus, 351-354 of p38, analyzing the three-dimensional structure of the protein kinase bound to the inhibitor, and designing an inhibitor of a protein kinase which also binds to amino acids corresponding to these amino acids of p38 by employing molecular modeling means.

The identification of ATP-binding site residues of the first protein kinase, which may be non-crystallizable, may be performed via protein alignment methods known to those skilled in the art and may be performed using one of many alignment algorithms. State of the art computer programs are available for aligning proteins having homologous sequences and/or structures. One example of a homology alignment program is PILEUP (Genetics Computer Group) which compares multiple sequences of related proteins and nucleotides and generates an alignment of these sequences for comparison, MULTALIN [Corpet, Nucleic Acids Res 16, 10881-90 (1988)]is another homology alignment program. For a nonlimiting summary of alignment algorithms, see Methods in Enzymology, 1990, 183:1-736.

The second protein kinase may be selected based on its three-dimensional structure being known, its implications in disease, and/or its ability to be crystallizable. Several protein kinases have been crystallized. These are cAPK [Knighton et al, Science 253, 407-413 (1991)], c-Src [Xu et al. Nature 385,595-602(1997)],CDK2 [Russo et al. Nature 382 325-331 (1996)], ERK2 [Zhang et al. Nature 367 704-711(1994)], CK1 [Longenecker et al. J. Mol. Biol 257; 618-631(1996)], and FGF-R[Mohammadi, et. al. EMBO J. 17; 5896-5904 (1998). The first protein kinase may be selected by its implication in disease and/or by searching a searchable database, such as GenBank, to identify amino acid sequences which share homology to the second protein kinase. In one embodiment, the first protein kinase which is selected from GenBank may map to a chromosomal locus implicated in a disease or may be implicated in a disease by another criteria known in the art.

The present invention may allow for the identification of inhibitors to protein kinases which have been and are being identified through the information from the genome sequencing efforts. For example, it is likely that many of the potential protein kinases which may be identified by a homology search of amino acids sequences in GenBank using a known protein kinase, will not be crystallizable. The chimeric protein kinases of the present invention may provide the ability to routinely crystallize chimeric protein kinases comprising amino acids (either the ATP binding site or the inhibitor binding site of the present invention which is outside the ATP binding site) from newly identified, unknown and/or non-crystallizable protein kinases and to design inhibitor molecules which bind to the inhibitor binding site.

The identification of non-ATP binding site residues of the second protein kinase can be performed by analyzing the X-ray crystal structure of the second kinase co-complexed with an inhibitor that binds to the ATP binding site or co-complexed with ATP. Standard techniques may be used to obtain the three-dimensional structure of the second kinase, including NMR and X-ray crystallography. For X-ray crystallographic determination of the three-dimensional structure, the second kinase can be crystallized by standard techniques known in the art and the crystals obtained can be subject to X-ray diffraction data collection using conventional equipment. The diffraction data obtained can then be used to solve the three-dimensional structure of the second kinase. The three-dimensional structure can then be analyzed using standard techniques in order to identify the non-ATP binding site amino acids.

The atomic, three-dimensional structure of a protein kinase according to the present invention may be determined by any means known in the art. Preferably, the atomic three-dimensional structure may be determined using molecular replacement. The term “molecular replacement” refers to a method that involves generating a preliminary model of the three-dimensional structure of the chimeric protein kinase crystals of the present invention whose structure coordinates are unknown prior to the employment of molecular replacement. Molecular replacement is achieved by orienting and positioning a molecule whose structure coordinates are known (in this case the previously determined second protein kinase) within the unit cell as defined by the X-ray diffraction pattern obtained from a chimeric protein kinase crystal whose structure is unknown so as to best account for the observed diffraction pattern of the unknown crystal. Phases can then be calculated from this model and combined with the observed amplitudes to give an approximate Fourier synthesis of the structure whose coordinates are unknown. This in turn can be subject to any of several forms of refinement to provide a final, accurate structure.

Any method known to the skilled artisan may be employed to determine the structure by molecular replacement. For example, the program AMORE (The CCP4 suite: Programs for computational crystallography, 1994, Acta Crystallogr. D. 50:760-763) maybe employed to determine the structure of an unknown chimeric protein kinase of the present invention +/− an inhibitor by molecular replacement using the second protein kinase coordinates which have been previously determined. For the structure determination of the inhibitory compound, the structure of the inhibitor, if known, may be obtained from the Cambridge Structural Database (Refcode TRCHST, <<http://www.ccdc.cam.ac.uk >>) which structure may be employed to define the stereochemical restraints used in the refinement with the program CNS (Brunger et al., 1998, Acta Crystallogr. D 54:905-921).

An initial model of the three-dimensional structure may be built using the program O (Jones et al., 1991, Acta Crystallogr. A 47:110-119). The interpretation and building of the structure may be further facilitated by use of the program CNS (Brunger et al., 1998, Acta Crystallogr. D 54:905-921).

For the determination of the chimeric protein kinase structure, if the space group of the chimeric protein kinase crystal is different from the second protein kinase crystals, molecular replacement may be employed using a known structure of the second protein kinase or any known protein kinase structure whose structure may be determined as described above. If the space group of the chimeric protein kinase crystals is the same, then rigid body refinement and difference Fourier may be employed to solve the structure using a known structure of the second protein kinase or any protein kinase with a known structure having the same space group.

Generating the three-dimensional structure of the second kinase is not a required step in the present invention. A known structure may be employed to design the chimeric protein kinase of the present invention. Alternatively, protein alignment methods can be used to determine the location of non-ATP binding site amino acids, as well as ATP binding site amino acids by determining regions of homology between protein kinases whose ATP binding site amino acids and non-ATP binding site amino acids have been mapped by a methodology known in the art.

For the purposes of further describing the three-dimensional structures of the present invention, the definition of the following terms is provided:

The term “β sheet” refers to two or more polypeptide chains (or β strands) that run alongside each other and are linked in a regular manner by hydrogen bonds between the main chain C═O and N—H groups. Therefore all hydrogen bonds in a beta-sheet are between different segments of polypeptide. Most β-sheets in proteins are all-parallel (protein interiors) or all-antiparallel (one side facing solvent, the other facing the hydrophobic core). Hydrogen bonds in antiparallel sheets are perpendicular to the chain direction and spaced evenly as pairs between strands. Hydrogen bonds in parallel sheets are slanted with respect to the chain direction and spaced evenly between strands.

The term “α helix” refers to the most abundant helical conformation found in globular proteins. The average length of an α helix is 10 residues. In an α helix, all amide protons point toward the N-terminus and all carbonyl oxygens point toward the C-terminus. The repeating nature of the phi, psi pairs ensure this orientation. Hydrogen bonds within an α helix also display a repeating pattern in which the backbone C═O of residue X (wherein X refers to any amino acid) hydrogen bonds to the backbone HN of residue X+4. The α helix is a coiled structure characterized by 3.6 residues per turn, and translating along its axis 1.5 Å per amino acid. Thus the pitch is 3.6×1.5 or 5.4 Å. The screw sense of alpha helices is always right-handed.

The term “loop” refers to any other conformation of amino acids (i.e. not a helix, strand or sheet). Additionally, a loop may contain bond interactions between amino acid side chains, but not in a repetitive, regular fashion.

Amino acid residues in peptides shall herein after be abbreviated as follows: Phenylalanine is Phe or F; Leucine is Leu or L; Isoleucine is Ile or I; Methionine is Met or M; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; and Glycine is Gly or G.

The term “positively charged amino acid” refers to any amino acid having a positively charged side chain under normal physiological conditions. Examples of positively charged amino acids are Arg, Lys and His. The term “negatively charged amino acid” refers to any amino acid having a negatively charged side chain under normal physiological conditions. Examples of negatively charged amino acids are Asp and Glu. The term “hydrophobic amino acid” refers to any amino acid having an uncharged, nonpolar side chain that is relatively insoluble in water. Examples of hydrophobic amino acids are Ala, Leu, Ile, Gly, Val, Pro, Phe, Trp and Met. The term “hydrophilic amino acid” refers to any amino acid having an uncharged, polar side chain that is relatively soluble in water. Examples of hydrophilic amino acids are Ser, Thr, Tyr, Asp, Gln, and Cys. The term “aromatic amino acid” refers to any amino acid comprising a ring structure. Examples of aromatic amino acids are His, Phe, Trp and Tyr.

As used here, the phrase “non-inhibitor binding site amino acids” refers to those amino acids which are not involved in the binding of an inhibitor to the protein kinase. Correspondingly, the phrase “inhibitor binding site amino acids” refers to those amino acids which are involved in the binding of inhibitor to the protein kinase, which may include amino acids which are physically close enough to an atom or atoms of an inhibitor and the atoms are of such a nature that they would form covalent or non-covalent bonds, such as hydrogen bonds or van der Waals or electrostatic, as well as amino acids which form the pocket in which the inhibitor binds but which may not be in close physical proximity with the inhibitor to form a non-covalent bond. Physical distances of less than 4 Å are required to form significant covalent or non-covalent interactions. The inhibitor binding site may be the ATP site, the inhibitor binding site disclosed herein which is outside the ATP binding site, or another inhibitor binding site.

The preparation of the chimeric protein kinase may be achieved by methods well known to those of skill in the art. For example, the inhibitor biding site amino acids of the second protein kinase may be substituted with the inhibitor binding site amino acids of the first protein kinase using site-directed mutagenesis, PCR, or other methods of altering the DNA, or a cDNA encoding the second protein kinase. The chimeric protein kinase may then be expressed by conventional recombinant DNA techniques (may be expressed in prokaryotic and/or eukaryotic cells, such as bacteria, yeast or insect cells, as described further below) and may be purified using conventional chromatography, including ion exchange, gel filtration, affinity chromatography

The nucleic acids encoding the protein kinases of the present invention may be subcloned into an expression vector to create an expression construct such that the resultant protein kinase molecule which is produced comprises a fusion protein wherein said fusion protein comprises a tag for ease of purification. As referred to herein, a “tag” is any additional amino acids which are provided in a protein either c-terminally, n-terminally or internally for the ease of purification, for the improvement of production or for any other purpose which may facilitate the goals of the present invention (e.g. to achieve higher levels of production and/or purification). Such tags include tags known to those skilled in the art to be useful in purification such as, but not limited to, his tag, glutathione-s-transferase tag, flag tag, mbp (maltose binding protein) tag, etc. Such tagged proteins may also be engineered to comprise a cleavage site, such as a thrombin, enterokinase or factor X cleavage site, for ease of removal of the tag before, during or after purification. Vector systems which provide a tag and a cleavage site for removal of the tag are particularly useful to make the expression constructs of the present invention.

A large number of vector host systems known in the art may be used to express the protein kinases of the present. Possible vectors include, but are not limited to, plasmids or modified viruses, but the vector system must be compatible with the host cell used. Examples of vectors include E. coli bacteriophages such as lambda derivatives, or plasmids such as pBR322 derivatives or pUC plasmid derivatives, e.g., pGEX vectors (Amersham-Pharmacia, Piscataway, N.J.), pET vectors (Novagen, Madison, Wis.), pmal-c vectors (Amersham-Pharmacia, Piscataway, N.J.), pFLAG vectors (Chiang and Roeder, 1993, Pept. Res. 6:62-64), baculovirus vectors (Invitrogen,Carlsbad, Calif.; Pharmingen, San Diego, Calif.), etc. The insertion into a cloning vector can, for example, be accomplished by ligating the DNA fragment into a cloning vector which has complementary cohesive termini, by blunt end ligation if no complementary cohesive termini are available or by through nucleotide linkers using techniques standard in the art. E.g., Ausubel et al. (eds.), Current Protocols in Molecular Biology, (1992). Recombinant vectors comprising the nucleic acid of interest may then be introduced into a host cell compatible with the vector (e.g. E. coli, insect cells, mammalian cells, etc.) via transformation, transfection, infection, electroporation, etc. The nucleic acid may also be placed in a shuttle vector which may be cloned and propagated to large quantities in bacteria and then introduced into a eukaryotic cell host for expression. The vector systems of the present invention may provide expression control sequences and may allow for the expression of proteins in vitro.

As indicated above, the chimeric protein kinase of the present invention is useful for identification of inhibitor molecules that bind to the inhibitor binding site of the first protein kinase. Accordingly, the present invention is directed to a method for identifying an inhibitor of a first protein kinase comprising making a chimeric protein kinase having an inhibitor binding site comprising amino acid residues of the first protein kinase which bind to the inhibitor amino acid residues of a second protein kinase which do not bind the inhibitor, crystallizing the resultant chimeric protein kinase by methods known in the art, obtaining X-ray crystallographic data from the crystallized chimeric protein by methods known in the art, determining the three-dimensional structure of the chimeric protein kinase from the X-ray crystallographic data, analyzing the three-dimensional structure of the chimeric protein kinase, designing an inhibitor compound which binds to the chimeric protein kinase using molecular modeling means known in the art, and determining whether the designed inhibitor compound inhibits the first protein kinase.

The inhibitor molecule may be designed by using a molecular modeling techniques to place a known inhibitor which inhibits the second protein kinase into the three-dimensional structure of the chimeric protein kinase. Because the inhibitor binding site is from the first protein kinase, reasons why the inhibitor cannot effectively inhibit the first protein kinase may be apparent, such as steric hindrances and otherwise other incompatibilities. Molecular modeling software may be employed for suggesting potential changes of the inhibitor molecule to identify an effective inhibitor that would bind to the chimeric protein kinase and, preferably the first protein kinase.

One of skill in the art may use one of several methods to screen chemical moieties to replace portions of the inhibitor so that binding to the chimeric protein kinase and/or the first protein kinase is optimized. This process may begin by side-by-side visual inspection of the three-dimensional structure of the second protein kinase bound to the inhibitor and the three-dimensional structure of the chimeric protein kinase and, as indicated above, by modeling the inhibitor of the second protein kinase into the three-dimensional structure of the chimeric protein kinase.

Modified inhibitors may be tested for their ability to bind to the chimeric protein kinase and/or the first protein kinase using software such as DOCK and AUTODOCK followed by energy minimization and molecular dynamics with standard molecular mechanics force fields, such as CHARMM and AMBER. The following nonlimiting list of computer programs may be useful in the present invention: (1) GRID [Goodford, J. Med. Chem. 28:849-857 (1985); available from Oxford University, Oxford, UK, (2) MCSS [Mrianker et al., Proteins: Structure, Function and Genetics 11:29-34 (1991)]; available from Molecular Simulations, Burlington, Mass., (3) AUTODOCK [Goodsell et al., Proteins: Structure, Function, and Genetics, 8:195-202 (1990)]; available from Scripps Research Institute, La Jolla, Calif., (4) DOCK [Kuntz et al., J. Mol. Biol. 161:269-288 (1982)]; available from University of California, San Francisco, Calif., (5) LUDI [Bohm, J. Comp. Aid. Molec. Design 6:61-78 (1992)]; available from Biosym Technologies, San Diego, Calif., (6) LEGEND (Nishibata et al, Tetrahedron 47:8985 (1991)]; available from Molecular Simulations, Burlington, Mass., (7) at LeapFrog; available from Tripos Associates, St. Louis, Mo.; GRAM, [Dunbrack et al., Folding & Design 2:27-42 (1997)], and (8) HOOK [Dunbrack et al., Folding & Design 2:27-42 (1997)]. Other molecular modeling techniques may also be employed in accordance with this invention, such as, but not limited to Cohen et al., J. Med. Chem. 33:883-894 (1990), and Navia et al., Current Opinions in Structural Biology 2:202-210 (1992).

The inhibitor molecule should bind with an affinity great enough to inhibit the ATP hydrolysis activity of the kinase by at least 3 fold and preferably 10 to 1000 fold. Additionally, inhibition ability of the inhibitor may be determined by the ability of the inhibitor to inhibit the ability of the protein kinase to phosphorylate a substrate by at least 3 fold and preferably 10 to 1000 fold. An inhibitor which binds to the protein kinase with a K_{d} and/or a Kᵢ of less than 1 μM is preferred. More preferably, the inhibitor will bind to the protein kinase with a K_{d} and/or a Kᵢ i of less than 100 nM. The determination of inhibitory activity by measuring ATP hydrolysis or phosphorylation of a substrate are well known to those in the art [Buechler and Taylor Biochemistry 27, 7356-61 (1988)]; Prowse et al. Biochemistry 39, 6258-66 (2000)]. In addition, the measurement of K_{d} and/or Kᵢ are also well known in the art [see Enzyme Structure and Mechanism, Second Edition, Alan Fersht, ed., W. H. Freeman and Company, New York (1985)].

Once an inhibitor has been designed and/or identified by the methods of the present invention, the efficiency with which the inhibitor binds and/or inhibits the chimeric protein kinase and/or the first protein kinase may be tested and further optimized by computational evaluation. For example, optimization may include eliminating any aspects of the inhibitor that may cause repulsive electrostatic interaction with the chimeric and/or first protein kinase. Such repulsive electrostatic interactions include repulsive charge-charge, dipole-dipole, and charge-dipole interactions. Preferably, the sum of all electrostatic interactions between the inhibitor and the chimeric protein kinase and/or the first protein kinase when the inhibitor is bound to the inhibitor binding pocket preferably make a neutral or favorable contribution to the enthalpy of binding. Computer programs useful for analyzing electrostatic charges include, but are not limited to, Gaussian 92, revision C [M. J. Frisch, Caussian, Inc., Pittsburgh, Pa. © 1992]; AMBER, version 4.0 [P. A. Kollman, University of California at San Francisco, ©1994]; QUANTA/CHARMM [Molecular Simulations, Inc. Burlington, Mass. ©1994]; and Insight II/Discover (Biosysm Technologies Inc., San Diego, Calif. ©1994). All the programs mentioned herein may be implemented using a computer such as a Silicon Graphics work station. Other hardware systems and software packages are known to those skilled in the art and may be used in accordance with the present invention.

The present invention is also directed to a protein kinase inhibitor binding site which is outside the ATP binding site and methods of use therefore. As indicated, the protein kinase inhibitor binding site of the present invention is outside the ATP binding site, which is a well known inhibitor binding site for protein kinases. In addition, the protein kinase inhibitor binding site of the present invention is in a region of less homology among protein kinases and therefore may allow for the identification of inhibitor molecules which display improved specificity.

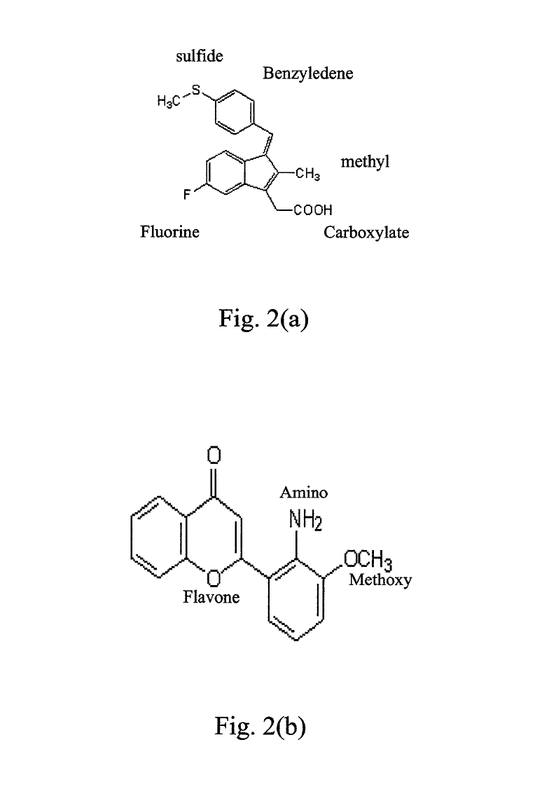

The inhibitor binding site of the present invention which is outside the ATP binding site comprises amino acids corresponding to a structural region of p38 wherein the region comprises linker L5 (residues 76 to 83) that joins helix C (residues 63-75) with β4 (residues 84-89), as well as the cross over connection, L7, (residues 106-109) and the C-terminus (βL6) (residues 310-336). The protein kinase inhibitor binding site of the present invention is at the hinge site between a helix rich domain of the protein kinase and a beta sheet rich domain of the protein kinase (referred to herein as the two domains of the protein kinase). The protein kinase inhibitor binding site of the present invention binds to, inter alia, the inhibitors sulindac sulfide {cis-5-flouro-2-methyl-1-[p-(methylsulfinyl)benzylidene]indene-3-acetic acid}(see FIG. 2(a) ), which inhibits cyclooxygenase and IKK-β and is a non-steroidal anti-inflammatory and PD98059 {1-(2′-amino-3′-methoxyphenyl)-oxanaphthalen-4-one}(see FIG. 2(c)) which is a flavonoid which binds specifically to and inhibits the activation of MEK1 by c-Raf and other upstream activators [see Alessi et al., J. Biol. chem. 270:27489-27494 (1995)] and is non-competitive with respect to ATP hydrolysis [Dudley et al., Proc. Natl Acad. Sci. USA 92:7686-7689 (1995).

The protein kinase inhibitor binding site of the present invention which lies outside the protein kinase ATP binding site was determined by analyzing the three-dimensional structure of p38 bound to sulindac sulfide and/or PD98059. See Example 1 below.

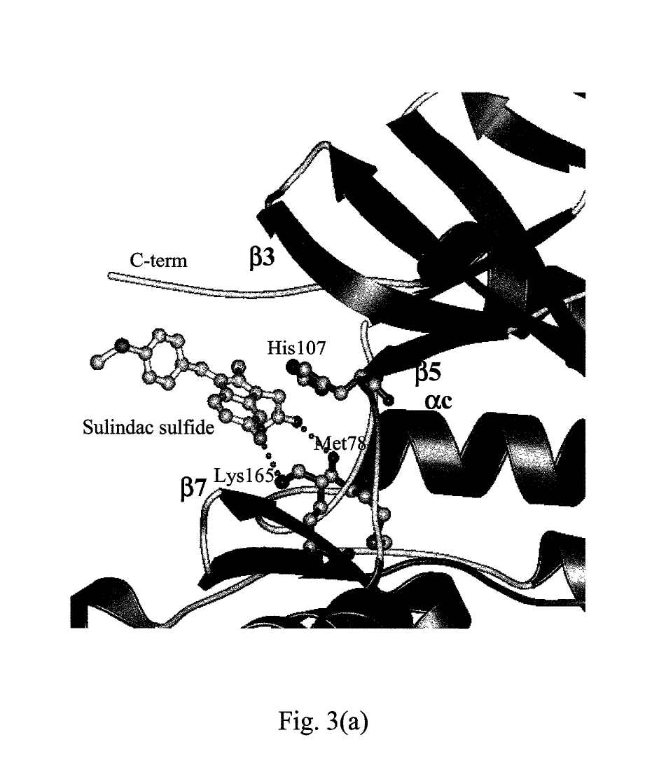

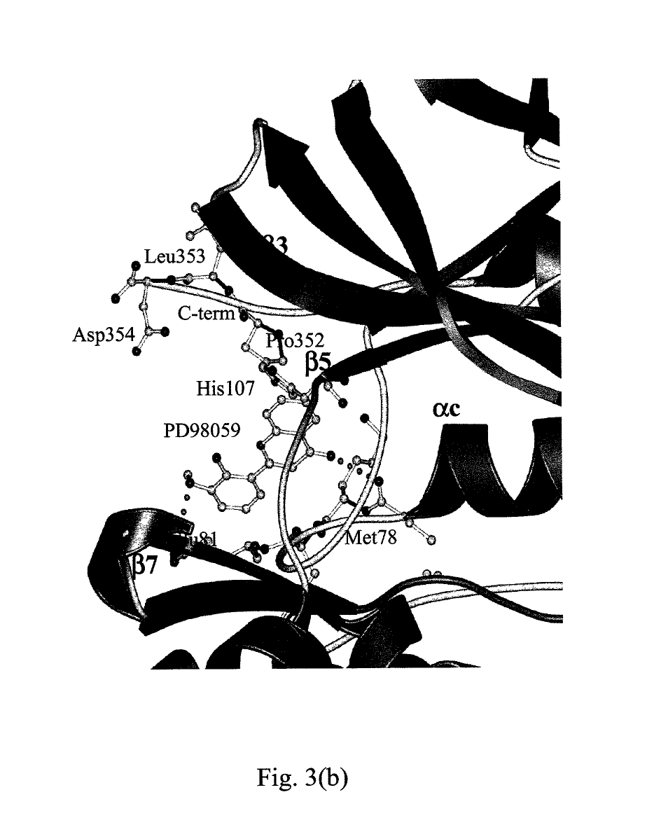

Sulindac sulfide includes an indene moiety, a carboxylate group moiety, a benzyledene moiety and a fluorine moiety (FIG. 2a). These moieties are in close proximity to amino acids in the protein kinase inhibitor binding site of the present invention which is outside the ATP binding site. For example, the indene moiety is held between His 107 and Lys 165; there are weak ring interactions between the six membered ring of the indene moiety and the pyrimidine ring in His 107; the carboxylate group moiety forms weak hydrogen bonds with p38 backbone atoms, specifically the carbonyl of Met78 (which is 3.36 Å away); His80 N and the carboxylate groups of Glu81 in the linker between helix C and β-4; the benzyledene moiety forms van der Waals contacts with the C-terminal amino acids 353 and 354 of p38; and the fluorine moiety, which is connected to the indene moiety, is hydrogen bonded (2.88 Å) to Lys165Nε (FIG. 3(a)).

PD98059 contains a flavone substituted with an amino group at the 2′ position, and a methoxy moiety at the 3′ position (See FIG. 2(b). PD98059 binds at the hinge point between the two protein kinase domains. It is flanked by the β sheets 3 and 7. The binding pocket is at the cross-over connection close to the C-terminal chain of the molecule. There is no ring stacking like that found in sulindac sulfide between the six membered ring of the indene and the pyrimidine ring of the His 107, but instead, the interaction is edge of ring to edge of ring. There are weak hydrogen bonding interactions between the carboxylate groups of Glu81 and the backbone atoms of Lys 79 and the flavone ring of PD98059. Weak van der Waals interactions occur between the flavone of PD98059 and the C-terminal amino acids 350-354 and between carbonyl of the flavone ring of the carbonyl of Met 78 (3.06 Å) (see FIG. 3(b)).

In another embodiment, the present invention provides a chimeric protein kinase which comprises inhibitor binding site residues (as defined by the protein kinase inhibitor binding site of the present invention which is outside the ATP binding site) from a first protein kinase and non-inhibitor binding site residues from a second kinase. In a preferred embodiment, the second kinase is p38 and the first kinase is selected from the group consisting of IKK-β, Map/ERK kinase, cJun, JNK, and MEK. The resultant chimeric protein kinase is preferably crystallizable.

In a particularly preferred embodiment, the chimeric protein kinase of the present invention comprises nearly all the amino acids of p38 with just the amino acids of IKK-β corresponding to the inhibitor binding site of the present invention which is outside the ATP binding site. This chimeric protein kinase has the following mutations in the p38 amino acide sequence: His107 of p38 to Tyr; Glu81 of p38 to Pro and Leu353 of p38 to Ala. In another embodiment of the invention, the chimeric protein kinase of the present invention comprises nearly all the amino acids of p38 with just the amino acids of Map/ERK corresponding to the inhibitor binding site of the present invention and comprises the amino acid residues of p38 with the following mutations: Lys79 of p38 to Asn; Glu81 of p38 to Pro; and the sequence in the C-terminus of p38 (351-356) of PPLDQE to THAASI. In a further embodiment of the invention, the chimeric protein kinase of the present invention comprises nearly all the amino acids of p38 with just the amino acids of JNK corresponding to the ATP binding and comprises the amino acid residues of p38 with the following mutations: Thr106 of p38 to Met; Tyr 35 of p38 to Gln; His107 of p38 to/Glu and Leu75 of p38 to Met.

The chimeric protein kinase is useful for identifying and/or designing inhibitor molecules of a protein kinase which do not bind to the ATP binding site of the protein kinase and which are preferably specific for the protein kinase of interest. The preferred binding affinity of the inhibitor is the same as the inhibitor described above. The inhibitory activity of the inhibitor may be determined by analyzing the ability of the inhibitor to block substrate phosphorylation, as described above.

The present invention is further directed to a method for identifying a protein kinase inhibitor which binds to the protein kinase inhibitor binding site of the present invention which is outside the ATP binding site. The method comprises crystallizing a protein kinase by methods known in the art; obtaining crystals comprising the protein kinase and PD98059 or sulindac sulfide (this may be accomplished by co-crystallizing the inhibitor PD98059 or sulindac sulfide with the protein kinase, or crystallizing the protein kinase alone and later soaking the crystals in a solution containing PD98059 or sulindac sulfide such that the PD98059 or sulindac sulfide enter the crystals and bind to the protein kinases), obtaining X-ray crystallographic data from the crystals comprising the protein kinase and PD98059 or sulindac sulfide by methods known in the art, determining the three-dimensional structure of the protein kinase and the PD98059 or sulindac sulfide from the X-ray crystallographic data, analyzing the three-dimensional structure of the P98509 or sulindac sulfide inhibitor binding site, designing an inhibitor compound which binds to the inhibitor binding site using molecular modeling means known in the art, and determining whether the designed inhibitor compound inhibits the protein kinase. In a preferred embodiment, the protein kinase is p38. In another preferred embodiment, the protein kinase is a chimeric protein kinase which comprises inhibitor binding site residues from a first protein kinase and non-inhibitor binding site residues from a second protein kinase. The first protein kinase may be non-crystallizable, or not easily crystallizable and the second protein kinase may be crystallizable. The resultant chimeric protein kinase is preferably crystallizable. In a further preferred embodiment, the chimeric protein kinase comprises amino acids Tyr98, Pro72, Ala 367 of IKK-β and the remainder from p38.

It is a further objective of the present invention to provide inhibitor molecules which inhibit a protein kinase by binding to the inhibitor binding site of the present invention which is outside the ATP binding site.

### EXAMPLES

### Example 1

P38 Crystal Formation

p38 crystals were obtained as previously described. See Wang et al., J. Biol. Chem. 6:1117-128 (1998). Purified rat p38α alternative splice form, NCBI data base entry AAK1541, was used to grow the crystals.

Crystals of p38 were soaked in sulindac sulfide and PD98059 to introduce the compounds into the crystals. Freshly dissolved 10 mM solutions of (1) sulindac sulfide in a buffer containing 50 mM NaCl and 0.1 M Hepes pH 7.4 (2) PD98059 in DMSO were prepared. The crystals were soaked in 18% PEG8000, 0.2 M Magnesium acetate, 0.1M Hepes pH7.0 and 0.1 mM to 2 mM of the inhibitor (Sulindac sulfide or PD98059) for 1-2 hours. A higher concentration of the compounds or longer time of soaking the crystals were damaged, indicating that the compounds really penetrated into the crystals.

### Example 2

Structure Determination

The structures of the p38 crystals soaked in either sulindac sulfide or PD98059 were solved using better than 2.7 Å resolution data and the structures refined to R-factors of 21% or better. The crystallographic parameters are listed in Table I below. The crystals were flash-frozen in liquid propane using 5-30% glycerol and maintained at −175° C. during the data collection. X-ray diffraction was collected on an Raxis-IC image plate with a rotating anode generator (Rigaku, Tokyo, Model RU300) using 1.54Å radiation. The data were integrated and scaled using the program HKL2000 (Otwinowski, Z., Oscillation data reduction program, in Data Collection and Processing, L. Sawyer, N. Issacs, and S. W. Bailey, Editors. 1993, Science and Engineering Council/Daresbury Laboratory: Warrington, United Kingdom. p. 56-62.). The crystals all had the same space group and cell dimensions as the native p38 crystals. The difference electron-density maps were calculated using the phases from the native p38 coordinates. The compounds were modified in Insight II from similar molecules obtained from the protein data bank {indomethicin for sulindac sulfide; and quercetin (3, 5, 6, 3′, 4′-pentahydroxy flavone) for PD98059. The corresponding protein data bank access codes are 4COX and 2HCK, respectively. These molecules were then fit into the electron density using the program O (Jones et al., 1991, Acta Crystallogr. A 47:110-119). Positional and B-factor refinements were carried out using X-PLOR (Brunger x-PLOR: A system for x-ray crystallography and NMR (Yale University, Dept. of Molecular Biophysics, New Haven, Conn. Version 3.85) and model building was done using the program O (Jones et al., 1991, Acta Crystallogr. A 47:110-119). Bulk solvent correction was applied at the final stage of refinement in X-PLOR. The backbone conformation of at least 80% of the amino acids is within the most favored regions of the Ramachandran plot with none in the disallowed regions as defined using the program PROCHECK (Laskowski et. Al. J. Appl. Crystallography 26 283-291 (1993)).

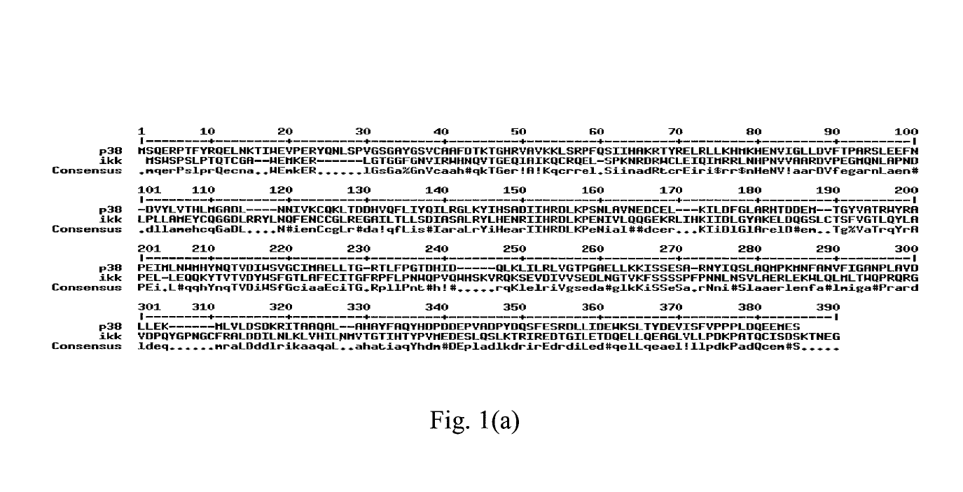

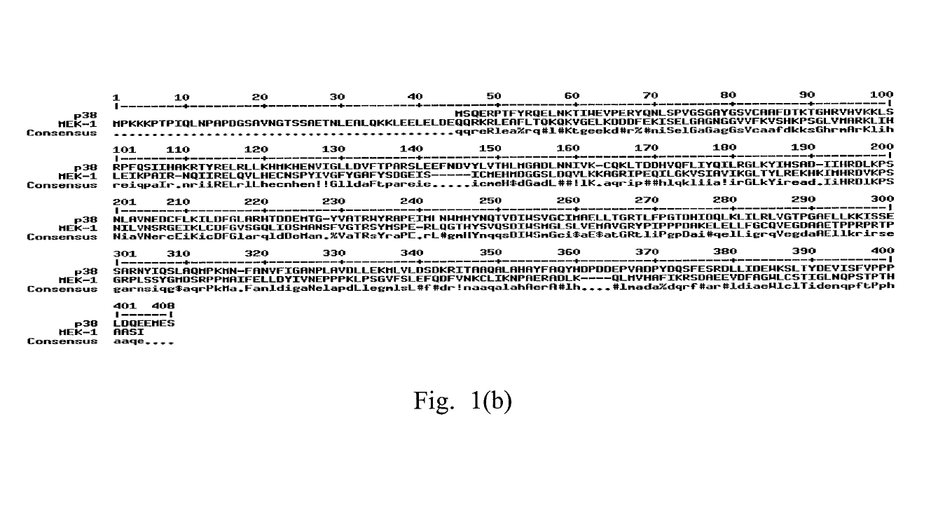

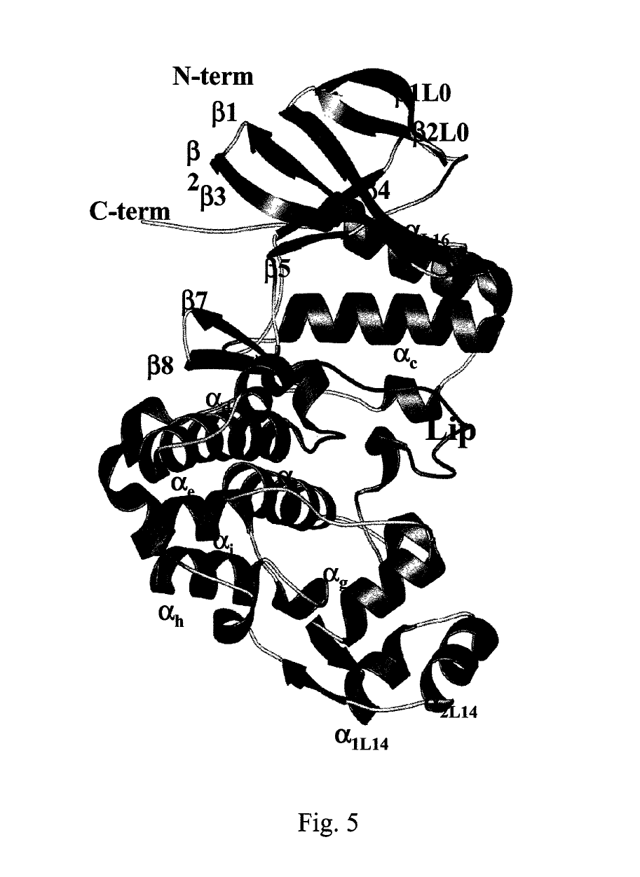

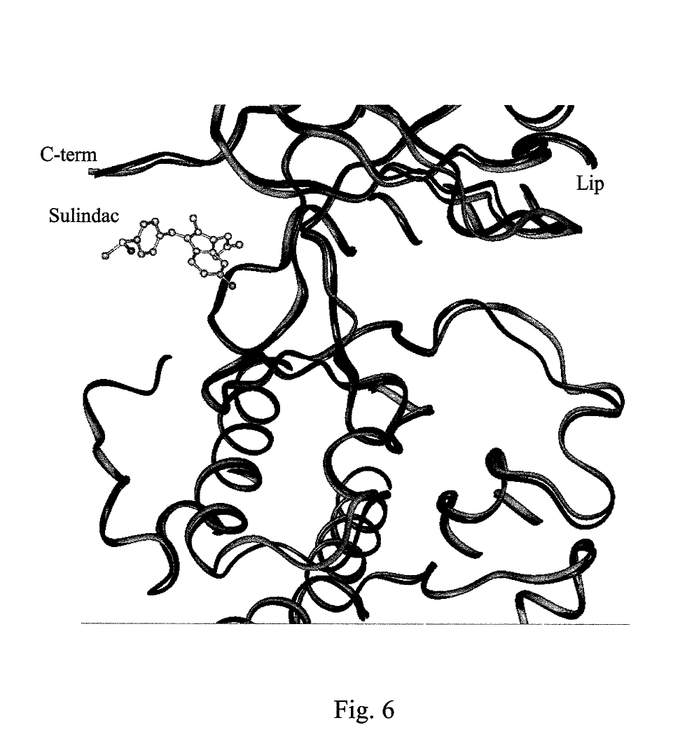

P38 and sulindac sulfide: Sulindac sulfide is an IKK-β inhibitor. The sequence identity of the protein kinase domain of IKK-β and p38 is about 30%. See FIG. 1(a). The N-termini start at about the same residue for both IKK-β and p38. Examination of the electron density for the p38 crystals soaked in sulindac sulfide revealed a strong peak at the hinge point between the two protein kinase domains (as described above). The sulindac sulfide was oriented and positioned in the electron density based on the strong density for the sulfur atom in the sulindac sulfide. The final refined model showed a strong density for the indene ring and the sulfide of the sulindac sulfide. Sulindac sulfide was found bound in a site outside the ATP binding site which is near the crossover connection of the protein kinase (FIG. 5 for definition of crossover connection). The indene moiety is held between the His 107 and Lys 165 residues of p38. There are weak ring stacking interactions between the six membered ring of the indene and the pyrimidine ring of the His 107. The carboxylate group in the sulindac sulfide forms weak hydrogen bonds with the backbone atoms, specifically carbonyl of Met78 (3.36 Å), His 80N and the carboxylate groups of Glu81 in the linker between helix C and β-4.(FIG. 5). The benzyledene forms van der Waals contacts with the C-terminal amino acids 353 and 354 of p38. The fluorine, which is connected to the indene, is hydrogen bonded to Lys 165 Nε(2.88 Å; see FIG. 3(a)).

p38 and PD98059: Using the information known for sulindac sulfide above, the location of PD98059 bound to p38 was determined by carefully examining the electron density maps for the crystals. The binding also occurs at the hinge point of p38 between the two protein kinase domains. PD98059 is flanked by β sheets 3 and 7(FIG. 5) The binding pocket, as with sulindac sulfide is at the cross-over connection close to the C-terminal chain of the molecule. There is no ring stacking like that found in the sulindac sulfide structure between the six membered indene ring and the pyrimidine ring of the His 107, instead, the interaction is in the two rings are edge to edge (There are weak hydrogen bonding interactions between the carboxylate groups of Glu81 and the backbone atoms of Lys 79 and the flavone moiety of PD98059. Weak van der Waals interactions between PD98059 and the C-terminal residues 350-353 and between the carbonyl oxygen of PD98059 and the carbonyl oxygen of Met78 were observed (3.06 Å; see FIG. 3(b)).

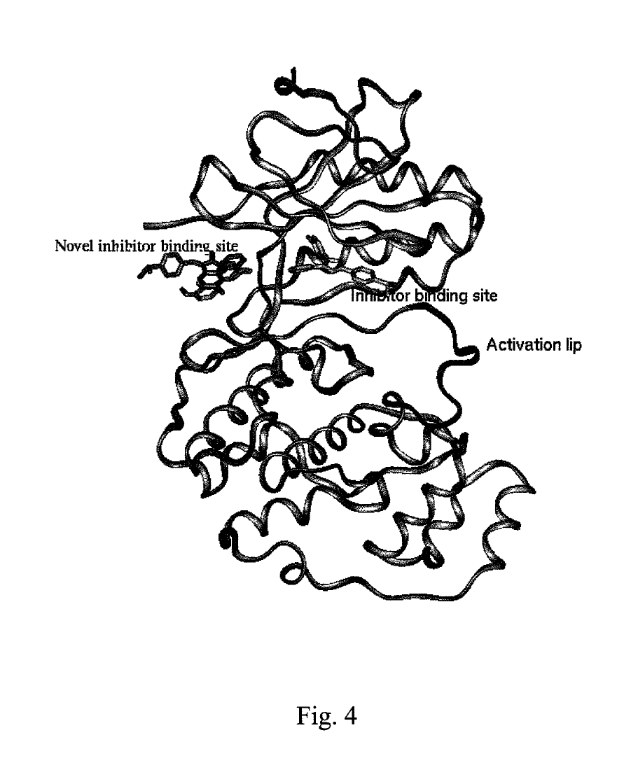

Binding site for sulindac sulfide and PD98059: The binding site in p38 for sulindac sulfide and PD98059 is at the hinge point between the two kinase domains. It is walled by the linker L5 (residues 76-83) that joins helix C (residues 63-75) with β4 (residues 84-89), the crossover connection (L7) (residues 106-109) and the C-terminus (βL1 6) (residues 310-336) (FIG. 5). This site is outside the catalytic site FIG. 4 shows the positions of the sulindac sulfide and PD98059 binding site of the present invention along with the the native ATP-competitive inhibitor binding site.

TABLE 1
X-ray Data Collection Parameters and Refinement Statistics

| | p38 + Sulindac | p38 + PD98059 |
|---|---|---|
| Diffraction data | | |
| Space group | P2₁2₁2₁ | P2₁2₁2₁ |
| Unit cell (Å) | a = 45.59 | a = 45.71 |
| | b = 85.01 | b = 85.54 |
| | c = 124.26 | c = 125.34 |
| Wavelength (Å) | 1.5418 | 1.5418 |
| Resolution (Å) | 2.5 | 2.2 |
| No. of measurements | 275364 | 264392 |
| Unique reflections | 14546 | 25889 |
| Completeness (%) (last shell) | 89.1(78.9) | 86.5(67.0) |
| R_{merge} (%) (last shell) | 5.6(32.4) | 4.8(37.5) |
| Refinement | | |
| Resolution (Å) | 20-2.6 | 20-2.4 |
| No. of reflections (F > 2σ) | 12448 | 15196 |
| R_{cryst}/R_{free} (%) | 21.7/25.9 | 21.1/24.1 |
| No. of waters | 98 | 91 |
| Ramachandran Plot: | | |
| Most favoured | 77.3 | 81.1 |
| Additional allowed | 19.2 | 17.0 |
| Generously allowed | 3.5 | 1.9 |
| Disallowed | 0 | 0 |

We claim:

1. A chimeric protein kinase having an inhibitor binding site comprising amino acid residues of a first protein kinase which bind an inhibitor and amino acid residues of a second protein kinase which do not bind the inhibitor.

2. The chimeric protein kinase of claim 1 wherein the first protein kinase is not crystallizable and the second protein kinase is crystallizable.

3. A crystal comprising a chimeric protein kinase having an inhibitor binding site comprising amino acid residues of a first protein kinase which bind an inhibitor and amino acid residues of a second protein kinase which do not bind the inhibitor.

4. The crystal of claim 3 wherein said crystal diffracts to greater than 5 Å.

5. A chimeric protein kinase comprising inhibitor binding site amino acid residues from a first protein kinase selected from the group consisting of IKK-β, Map/ERK, JNK, GSK-3, Akt, NIK and MEK and non-inhibitor binding site amino acid residues of a second protein kinase selected from the group consisting of p38 and ERK2, Src, CAPK, CK1, EGF-S, CDK2 and FGF-R.

6. The chimeric protein kinase of claim 5 wherein the inhibitor binding site is an ATP binding site.

7. The chimeric protein kinase of claim 5 wherein the inhibitor binding site is a non-ATP binding site.

8. The chimeric protein kinase of claim 7 wherein the second protein kinase is p38 which comprises inhibitor binding site residues of the protein first protein kinase.

9. The chimeric protein kinase of claim 8 wherein the first protein kinase is IKK-Å.

10. The chimeric protein kinase of claim 9 wherein p38 comprises amino acid changes of His107 to Tyr, Glu81 to Pro and Leu353 to Ala.

11. The chimeric protein kinase of claim 8 wherein the first protein kinase is Map/ERK.

12. The chimeric protein kinase of claim 11 wherein p38 comprises amino acid changes of Lys79 to Asn, Glu81 to Pro and the C-terminal sequence PPLDQE to THAASI.

13. The chimeric protein kinase of claim 8 wherein the first protein kinase is JNK.

14. The chimeric protein kinase of claim 13 wherein p38 comprises amino acid changes of Thr106 to Met, Tyr35 to Gln, His107 to Glu, and Leu75 to Met.

15. The chimeric protein kinase of claim 8 wherein the first protein kinase is MEK

16. The chimeric protein kinase of claim 15 wherein p38 comprises amino acid changes of Lys79 to Asn, Glu81 to Pro and the C-terminal sequence PPLDQE of p38 is changes to THAASI.

17. The chimeric protein kinase of claim 8 wherein the first protein kinase is GSK-3.

18. The chimeric protein kinase of claim 15 wherein p38 comprises amino acid changes of Lys79 to Asp, Glu81 to Cys, His107 to Asp and the C-terminal sequence PPLDQE to PHARIQ.

19. The chimeric protein kinase of claim 8 wherein the first protein kinase is Akt.

20. The chimeric protein kinase of claim 19 wherein p38 comprises the amino acid changes of Lys79 to Arg, Glu81 to Pro, His107 to Tyr and the C-terminal sequence PPLDQE to FPQFSV.

21. The chimeric protein kinase of claim 8 wherein the first protein kinase is NIK.

22. The chimeric protein kinase of claim 21 wherein p38 comprises amino acid changes of Lys79 to Arg, Glu81 to Val, His107 to Asn and the C-terminal sequence PPLDQE to TLAVKE.

23. A crystal comprising a chimeric protein kinase which comprises inhibitor binding site residues of a first protein kinase selected from the group consisting of IKK-β, Map/ERK, MEK, JNK, GSK-3, AKT and NIK and non-inhibitor binding site residues of a second protein kinase selected from the group consisting of p38, ERK2, Src, CAPK, CK1, EGF-R, CDK2 and FGF-R.

24. The crystal of claim 23 wherein said crystal diffracts to greater than 5 Å.

25. A method for identifying inhibitor molecules capable of affecting the activity of a first protein kinase comprising:
a) preparing a chimeric protein kinase comprising inhibitor binding site residues of the first protein kinase and non-inhibitor binding site residues of a second protein kinase wherein said chimeric protein kinase is cystallizable;
b) growing a crystal of said chimeric protein kinase;
c) solving the structure of said crystal using X-ray crystallography methods; and
d) using said structure to design inhibitor molecules capable of affecting the activity of the first protein kinase.

26. The method of claim 25 wherein the first protein kinase is selected from the group consisting of IKK-β, Map/ERK, JNK, MEK, GSK-3, Akt and NIK.

27. The method of claim 25 wherein the second protein kinase is selected from the group consisting of p38, ERK2, Src, CAPK, CK1, EGF-R, CDK2 and FGF-R.

28. The method of claim 25 wherein the inhibitor binding site is an ATP binding site.

29. The method of claim 25 wherein the inhibitor binding site is a non-ATP binding site.

30. The method of claim 29 wherein the inhibitor binding site is selected from the group consisting of PD98059 binding site and suldinac sulfide binding site.

31. The method of claim 25 wherein the first protein kinase is IKK-β and the second protein kinase is p38.

32. A protein kinase inhibitor binding site whose amino acid sequence corresponds to an amino acid sequence of, and has three-dimensional structural homology to, a protein kinase domain starting with linker L5 (residues 76-83) that joins helix C (residues 63-75) with β4 (residues 84-89), the crossover connection (L7) (residues 106-109) and ending at a C-terminus (βL16) (residues 310-336), wherein said domain is described to according to residues of p38.

33. The protein kinase inhibitor binding site of claim 32 wherein the protein kinase domain is derived from a protein kinase selected from the group consisting of p38, IKK-β, Map/ERK, JNK, MEK, GSK-3, Akt and NIK.
